(19) United States
(12) Patent Application Publication
(10) Pub. No.: US 2014/0316397 A1
(43) Pub. Date: Oct. 23, 2014

(54) Protective Sheath for Surgical Laser Fiber

(71) Applicant: Joe Denton Brown, Panama City, FL (US)

(72) Inventor: Joe Denton Brown, Panama City, FL (US)

(21) Appl. No.: 14/218,407

(22) Filed: Mar. 18, 2014

Related U.S. Application Data
(60) Provisional application No. 61/787,599, filed on Mar. 15, 2013.
(60) Provisional application No. 61/819,900, filed on May 6, 2013.
(60) Provisional application No. 61/824,755, filed on May 17, 2013.

(51) Int. Cl. A61B 18/24

(52) U.S. Cl. CPC A61B 18/24
USPC 606/15

(57) ABSTRACT

A protective sheath of the type used to protect a scope during insertion of a fiber into the scope may include holes for improved fluid flow and for ease of sterilization, reinforcement, a luer connector to position the sheath relative to a scope and to enable use of a seal, pin vise or other device to secure the fiber to sheath during insertion or withdrawal of the sheath from the scope, and/or markings to facilitate positioning of the sheath relative to the scope and/or trimming of the fiber.

This application claims the benefit of U.S. provisional patent application Ser. Nos. 61/824,755, filed May 17, 2013, 61/819,900, filed May 6, 2013, and 61/787,599, filed Mar. 15, 2013.

### BACKGROUND OF THE INVENTION

1. Field of the Invention

This invention relates to a device and method for delivering therapeutic light to a tissue, and in particular to an optical fiber arrangement in which a protective sheath is placed over the entire length of the fiber prior to insertion into an endoscope that guides the fiber to a treatment site. The protective sheath prevents mechanical damage to working channel of the endoscope during insertion of the fiber, insulates the fiber from surrounding cooling fluids, and may serve as an indicator of overheating that enables early detection of excess heating or burning of tissues or equipment at the treatment site.

2. Description of Related Art

The most important function of a protective sheath of the type with which the present invention is concerned is to allow the advancement of the relatively sharp-edged laser fiber tip through the ureteroscope without damaging the inner wall of the scope's working channel, thereby preventing expensive repairs to the scope. Previous sheaths had the disadvantage of a relatively thick wall or larger outer diameter, causing fluid flow through the working channel around the sheath/fiber assembly to greatly decrease, to the point where physicians refused to use the sheath due to reduced vision resulting from the reduced flow.

The need for a relatively thin-walled protective sheath is discussed in the inventor's copending U.S. patent application Ser. No. 13/127,911, filed May 5, 2011 (based on PCT Appl. No. PCT/US2009/006021) and the inventor's copending PCT Appl. No. PCT/US2009/006021, filed Nov. 6, 2009. The present invention provides improvements to the protective sheaths described in the inventor's copending applications/publications.

### SUMMARY OF THE INVENTION

It is accordingly an objective of the invention to provide various improvements to a protective sheath that surrounds a laser delivery fiber during insertion of the fiber into a surgical device, such as an endoscope.

A first improvement is to provide holes in the protective sheath to allow the physician to maintain adequate flow both through and around the sheath, thereby permitting the sheath to have an internal diameter (ID) that is close to the maximum outer diameter (OD) of the buffer of the optical fiber, In another version, a larger ID may be provided to allow for the introduction of larger diameter devices such as a 2.4 Fr basket. In this case, the holes provide a significant amount of additional flow through the sheath when the smaller diameter laser is used through the larger sheath.

This first improvement of providing holes in the sheath is also useful for sterilization of the sheath/fiber assembly. The holes allow for a much shorter path for the EtO sterilizing gas to reach all portions of the sheath/fiber assembly, thereby allowing a less robust (and cheaper) sterilization cycle to be used or providing a larger margin of safety to prevent occasional sterilization failures.

A second improvement, which may be used together with or separately from the first improvement, is to provide for the inclusion of a luer connector at the proximal ends of the sheath to precisely position the distal end of the sheath relative to the scope and/or to allow for attachment of a seal or pin vise if the physician desires to lock the position of the fiber to the sheath during insertion, use, or removal of the fiber/sheath assembly.

A third improvement, which may also be used separately from or together with each of the first two improvements, is to provide visible markings on the sheath for use as trim indicators. Additionally, markings on the fiber will enable the physician to correctly and easily position the fiber just recessed within the tip of the sheath during insertion or removal of the sheath from the working channel of the introducer or scope. The markings on the fiber are not limited to a single set, but rather may include multiple sets at both the distal and proximal ends of the fiber that can be used in case it is necessary to re-strip the fiber. A fourth improvement is the addition of a sealing mechanism that allows the physician to lock the fiber's position relative to the sheath and also seals the pressurized zone inside the working channel from the unpressurized zone outside of the working channel thereby preventing leakage from the working channel. This sealing mechanism can take several forms. A Touhy-Borst connector that has a male Luer lock connector can be locked to the female Luer lock end of the universal connector provided with the sheath that is already locked to the ureteroscope. This connector can then be tightened onto the laser fiber to lock relative positions and provide a leak proof seal. Alternatively a pin vise may be locked both to the universal Luer lock connector and then locked onto the laser fiber providing a leak proofseal. A third sealing mechanism consists of a septum-type seal that is pierced by the laser fiber or other instrument in the working channel and snaps easily over the sheath's female Luer lock connector already attached to the working channel. When the septum is pierced by the laser fiber, the flexible portion of the septum seals against the fiber preventing leakage past the seal, yet also locks the fiber's position relative to the sheath. This seal has the advantage that it provides easy adjustment of the laser fiber tip position relative to the end of the sheath. The physician can make this adjustment with one hand, instead of the two hands that are required with the first two sealing options. Another version has a connector that allows the position of the sheath to be adjusted relative to the working channel of the scope by the physician.

These and other improvements will be described in greater detail below in connection with the accompanying drawings, which show preferred embodiments of the invention.

### BRIEF DESCRIPTION OF THE DRAWINGS

FIG. 1 is a plan view of a sheath with holes for enhanced fluid flow and sterilization, and a luer adapter to allow locking the sheath to an endoscope working channel.

FIG. 2 is a plan view of the sheath of FIG. 1 with a locking device to hold the position of the fiber relative to the sheath.

FIG. 3 is a plan view of a sheath and fiber with a position indicator.

FIG. 4 is a plan view of the sheath and fiber of FIG. 3 with a pin vise to lock the fiber position.

FIG. 5 is a plan view of a sheath arrangement with a dilator and a coating for pull-in or fiber breakage detection.

FIG. 6 is a plan view of one embodiment of the fiber tip and sheath position relative to the end of the endoscope when the fiber is ready to treat tissue. FIG. 7 is an exploded plan view of one embodiment of the universal Luer lock connector, seal and marking on fiber relative to the Luer lock connector provided on the proximal end of the working channel of the endoscope.

FIG. 8 is an actual photo of one embodiment of the universal Luer lock connector, seal and marking on fiber relative to the Luer lock connector.

FIG. 9 is a plan view of one preferred embodiment of what the two sets of marks will look like on a laser fiber.

### DETAILED DESCRIPTION OF THE PREFERRED EMBODIMENTS

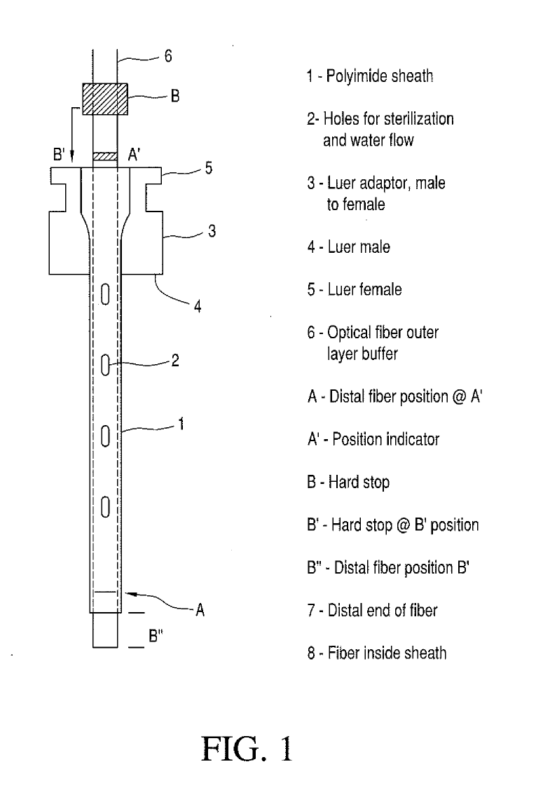

As shown in FIG. 1, the optical fiber buffer (6) is surrounded by a very thin-walled polyimide sheath (1) that performs several functions. The most important function of this sheath is to provide a protective sheath that allows the advancement of the relatively sharp-edged laser fiber tip through the ureteroscope without damaging the inner wall of the working channel, thereby preventing expensive repairs to the scope. Previous sheaths had such a thick wall and/or a large diameter that fluid flow through the working channel around the sheath/fiber assembly was greatly decreased, to the point where the physicians refused to use the sheath due to reduced vision due to reduced flow. The inner diameter (ID) of the sheath in this invention is very close to the maximum outer diameter (OD) of the buffer of the optical fiber (6) and the wall is very thin, thus providing maximum room for flow. Another design variation utilizes a larger ID that allows for the introduction of larger diameter devices such as a 2.4 Fr basket. In this case the holes (2) in the sheath (1) allow the physician to maintain adequate water or fluid flow both through and around the sheath that would not be available with a larger OD sheath without holes. Another potential use for this sheath is to provide for the injection of an addition fluid or gas at the very tip of the fiber.

A liquid dye that absorbs a laser wavelength extremely well could be injected through the fiber sheath to enhance the stone breaking properties of a given laser wavelength. Dye could be injected just prior to laser pulse through the coaxial sheath to ensure maximum concentration exactly at the fiber tip and also to minimize dye concentrations in other areas away from the fiber tip where the dye may reduce visibility of the physician. Dye may be able to be attached to another compound that has an affinity for and sticks to stones which will also help ensure a high concentration of dye very near the stone and minimize loss of visibility. A high viscosity dye may also be utilized to help maintain a high concentration near the stone and reduce tendency to become rapidly diluted in the irrigating flow. This technique may allow cheaper diode laser wavelengths such as 1470 nm to be used to break stones. The sheath connector that has the capability to adjust the position of the sheath relative to the scope will allow the dye to be injected immediately adjacent to the stone.

In addition to providing enhanced fluid flow, the holes (2) facilitate sterilization. Having an extremely small clearance between the ID of the sheath and the OD of the fiber over a very long length presents a major challenge to sterilizing this product. The addition of one or more small holes (2) to the sheath along the length of the sheath provides a greatly enhanced access of the sterilizing gas/agent to the entire length of the sheath/fiber assembly. In addition, these holes may be utilized in some designs to also provide fluid flow through the space between the fiber and sheath to augment the flow around the OD of the sheath.

FIG. 1 also shows a universal luer lock connector (3) attached to the reinforced section of the sheath. This universal luer lock (3) has a male luer connector (4) at its distal end to allow the physician to lock the sheath to the female luer lock at the entrance to the ureteroscope's working channel. When the luer lock is attached to the scope, the sheath will be precisely positioned extending approximately 2 mm (or any desired distance) from the end of the ureteroscope's working channel. The female luer lock connector (5) at the proximal end allows for subsequent attachment of a pin vise Touhy-Borst or a septum-type seal if the physician desires to lock the position of the fiber to the sheath during insertion, use or removal of the fiber/sheath assembly. This seal also functions to prevent leakage out of the working channel.

In order to assist the physician in positioning the distal end of the fiber, at least one position indicator A′ may be provided, as also shown in FIGS. 1, 7, 8 and 9. The correct position (A) of the fiber tip relative to the distal end of the sheath during insertion or removal of the sheath/fiber assembly from the scope's working channel is established when the position indicator (A′) is correctly positioned flush to the proximal entrance to the sheath. This ideal fiber tip position (A) may be, in the case of a urological application, approximately 1-2 mm recessed inside the sheath.

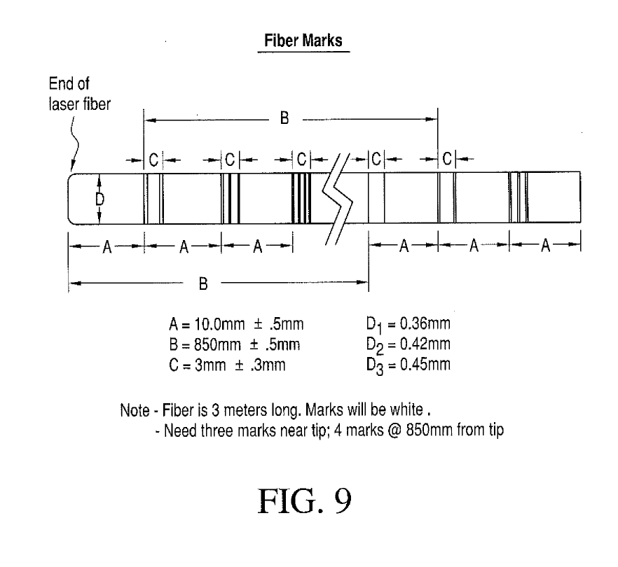

The width of this mark may be a precise width that will allow the front edge of the mark to be lined up at the entrance to the working channel of the scope to indicate the recessed fiber tip position, while the rear edge of the mark may be used to provide an indicator of the approximate position of the fiber tip when it is in an extended position approximately 1.5 mm in front of the sheath ready to treat tissue. This allows the physician to confidently extend the fiber out of the sheath without looking into the scope. Then when he does look into the scope, he can fine-tune the position of the already visible fiber tip to his individual preferred treatment extension length. To make provision for the event of the fiber needing to be re-cleaved during a procedure, a series of matched marks can be printed on the laser fiber at the distal tip and at the entrance to the scope as detailed above. These two sets of marks will be precisely positioned so that the fiber can be stripped and re-cleaved at the distal mark allowing the corresponding second mark to be used to position the fiber precisely at the entrance to the working channel of the scope ensuring that the re-cleaved fiber tip will again be positioned 1 mm recessed into the sheath during insertion and removal from the scope. The buffer stripping equipment provided with the fiber for re-cleaving will be designed to function with the distal marks to ensure correct placement when utilized with the corresponding proximal mark. Double, triple, etc., sets of marks may be provided to allow for a corresponding number of re-cleaving operations. FIG. 9 demonstrates one possible version of matching sets of marks.

Distance (A) is based on physician feedback as to the average or maximum length of fiber that is removed during each re-cleaving operation, e.g. 10 mm.

Distance (B) is based on the length of the sheath designed for the working channel of a particular scope brand and/or model, e.g. 850 mm for the Storz ×2 flexible ureteroscope. This length of the sheath will accommodate the variability in scope length of about ±2 mm by centering its target length at the point where the sheath tip is flush with the scope's tip or about 1 mm recessed into the scope. This will ensure that the sheath does not extend too far beyond the tip of the scope. Although the ideal placement of the sheath has the sheath just visible in the field of view, which is about 1 to 1.5 mm beyond the scope's tip, the sheath can be positioned just out of the field of view with no reduction in protection of the working channel's lining.

Distance (C) is based on the difference between the recessed fiber position (about 1 to 1.5 mm recessed into the sheath) and the protruding fiber position after it has been advanced to treat stones or tissue (about 1 to 2 mm in front of the end of the sheath). Therefore, this distance (C), approximately 2 to 3.5 mm, will allow the physician to use the front edge to indicate recessed position for sheath insertion/removal while the rear edge of mark will indicate the protruding position where the fiber is ready for use. The number of marks may be set to allow three re-cleaving procedures for a single use disposable, while a significantly larger number of marks may be provided for a multiple use fiber. The number of marks may be used to help ensure that a fiber is not used more than prudent number of times. Marks can be matched using a plurality of lines, different colors or shapes to enable the physician to determine which external mark to use after each re-cleaving operation.

Finally, as shown in FIG. 1, a hard stop (B) can be provided to prevent the laser fiber from extending too far past the end of the sheath as a safety mechanism to prevent damage to non-target tissue. The fiber can be advanced into the sheath until the hard stop (B) reaches the position (B′) where it is physically prevented from advancing any further due to its larger diameter not fitting into the sheath. The maximum advance of the distal tip of the fiber is indicated at B″.

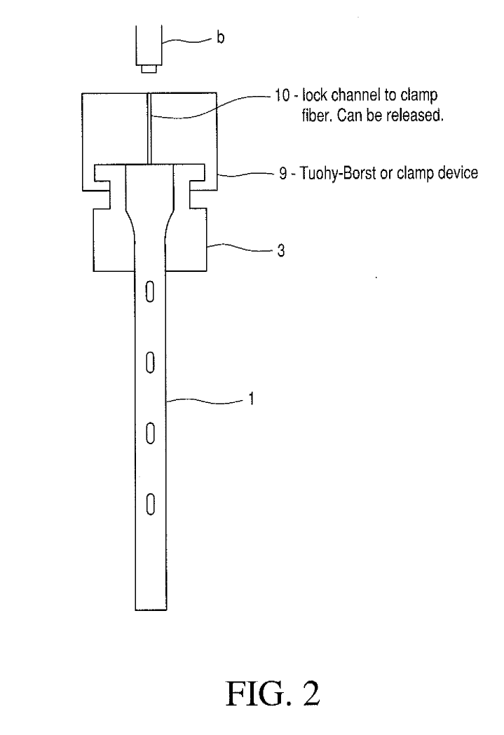

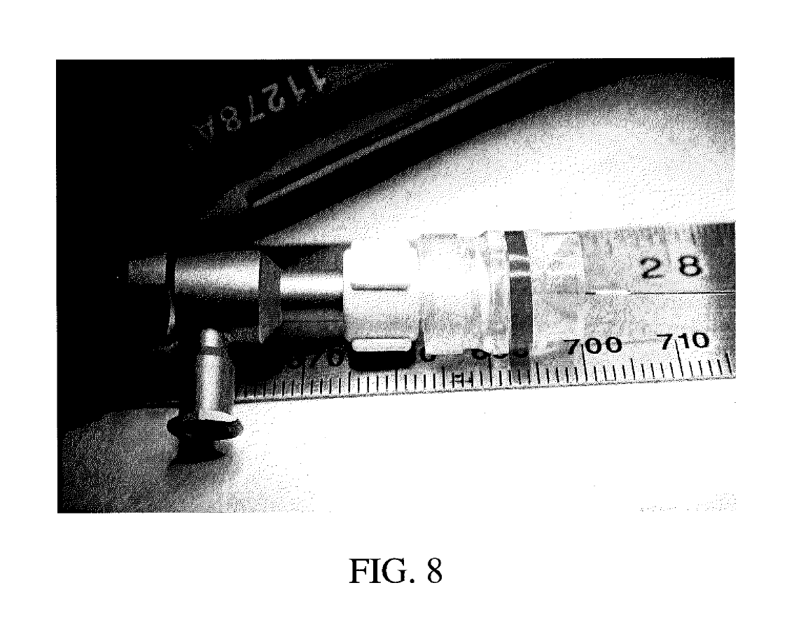

The arrangement shown in FIG. 2 differs from that of FIG. 1 in that a Touhy-Borst (9), a septum-type seal (11 in FIGS. 7 and 8), or other clamping device such as a pin vise is used to lock the fiber to the sheath during insertion, use or removal of the fiber/sheath assembly. The channel (10) where the fiber is clamped is also shown in FIG. 2.

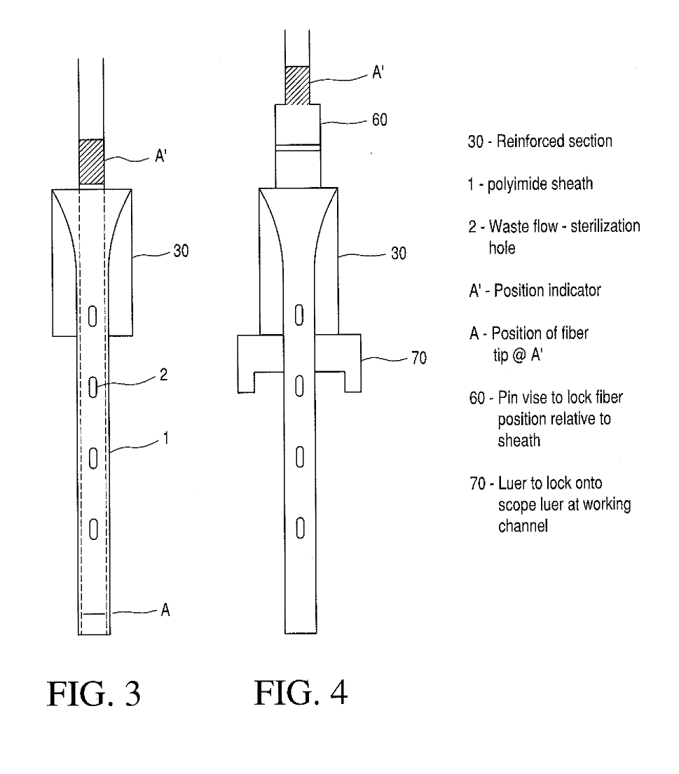

FIG. 3 shows a variation of the arrangement of FIG. 1, which also includes a very thin-walled polyimide sheath (1) with holes (2) and position indicator A′ corresponding to those of FIG. 1. However, the sheath of this embodiment is further provided with a reinforced section (30) at the proximal end of the sheath, which provides the following advantages. The primary advantage is that it provides a handling means for the physician to handle the extremely fragile polyimide sheath. This prevents kinking and/or breakage during insertion and use of the sheath/fiber assembly. The reinforced section (30) also has a funnel-shaped tip that enhances visibility and facilitates the insertion of the fiber tip into the sheath. Since the reinforced section (30) is short and does not extend past the irrigation side port, it does not reduce the flow around the sheath. A slanted cut on the proximal end of the polyimide sheath (1) minimizes the potential for the advancing fiber to catch on the small ledge formed at the proximal end of the sheath inside the reinforced portion.

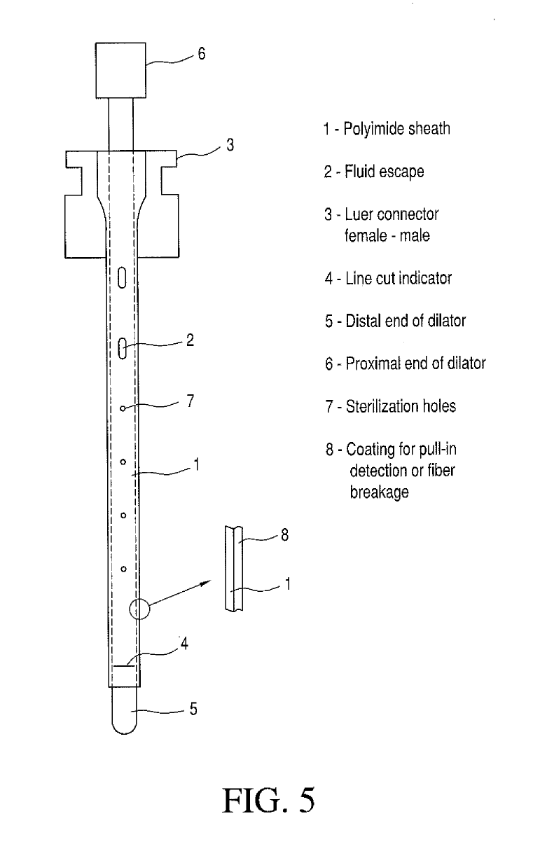

FIG. 4 shows a modification of the arrangement of FIG. 3 that includes a pin vise (60) to lock the fiber position relative to sheath. Some physicians may prefer to utilize a pin vise to lock the fiber to the sheath to ensure that they do not move relative to each other during insertion and removal from the working channel of the ureteroscope or other scope. In the case of a urological application, the pin vise (60) may be easily repositioned and re-locked after the fiber has been advanced from its position inside the sheath into the field of view adjacent to the target tissue. The fiber and sheath can be locked together at the desired recess position to allow the physician to take the assembly directly from the package and introduce it safely into the ureteroscope's working channel without having to manually insert the fiber into the sheath or check the position of the fiber relative to the sheath thereby simplifying the procedure.

The arrangement of FIG. 4 also includes a luer lock (70) attached to the reinforced section (30) of the sheath This universal luer lock (70) allows the physician to lock the sheath to a female luer lock at the entrance to the ureteroscope's working channel. When this luer lock is attached to the scope, the sheath will be precisely positioned extending approximately 2 mm from the end of the ureteroscope's working channel. However, a modified mechanism can be provided that allows the physician to adjust the position of the sheath relative to the scope.

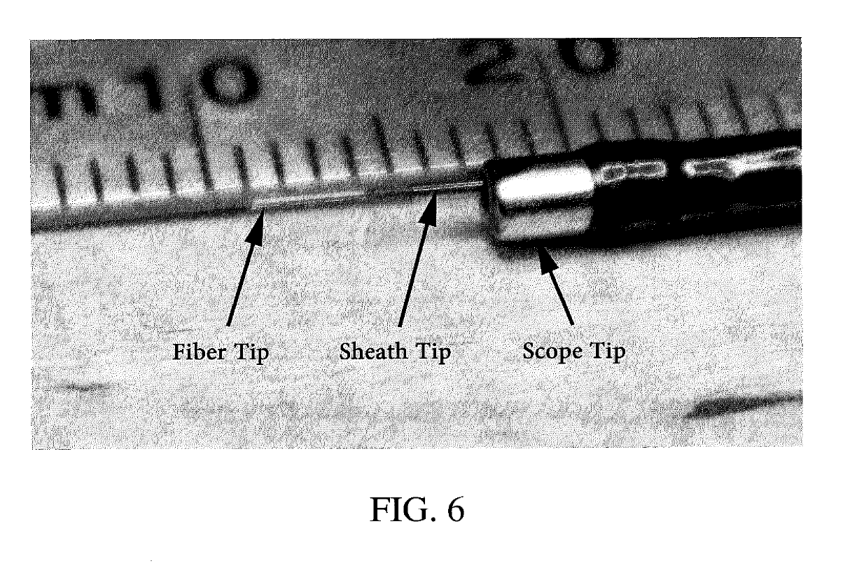

FIG. 5 shows a variation of the sheath (1) of FIG. 1, with an additional line cut indicator or indicators (4). The line cut indicator(s) (4) indicate where the physician should trim the sheath prior to insertion based on specific ureteroscope/connector combinations.

The arrangement of FIG. 5 further includes a dilator with proximal end (5) and distal end (6). The blunt dilator may be provided to allow insertion of the sheath prior to the insertion of the fiber. The distal blunt end (5) of the dilator would extend approximately 1 mm past the end of the sheath to prevent damage to the inner lining of the scope's working channel. The distal end (6) of the dilator may be provided with a male luer lock connection that would allow it to lock to the sheath during insertion. The dilator provides stiffening of the sheath to prevent kinking and damage to the sheath during insertion and removal from working channel.

The sheath (1) shown in FIG. 5 further includes a coating (8) for detection of fiber breakage or pull-in to sheath. The coating (8) is layered into or on the sheath to provide a distinct signal that enables the safety mechanism detector to differentiate between standard stone treatment pulses and pulses that occur if the fiber breaks or if the fiber is accidentally pulled into the sheath. If either of these occurs, the safety mechanism will recognize the distinct signal and shut the laser down prior to any damage to the ureteroscope's working channel. There are many ways that this coating may work. One instance is incorporation of Holmium coating that is activated by the Holmium laser that is treating the stones. When it is activated by that wavelength it will re-emit a much longer signal that can be readily differentiated from the Holmium treatment pulse and activate the safety shutdown.

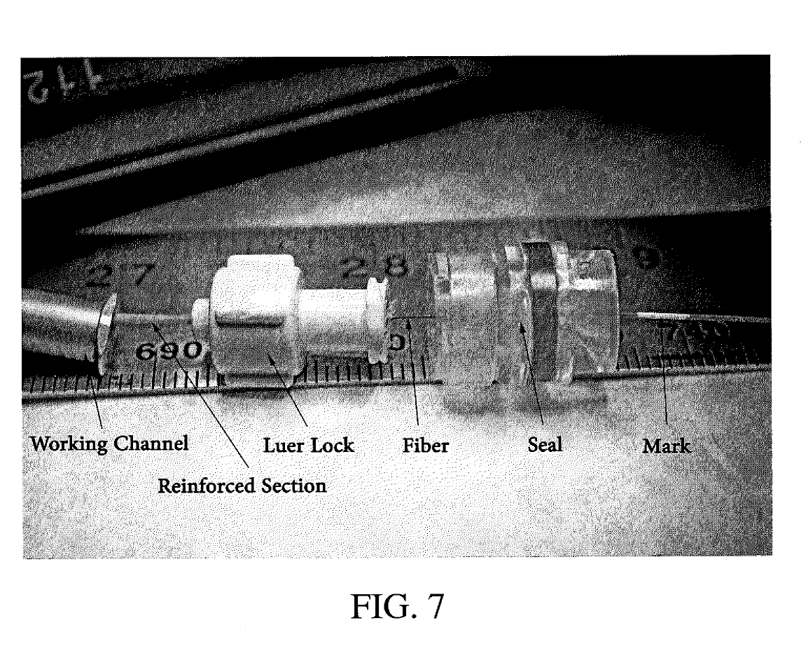

FIG. 6 demonstrates the sheath and fiber position relative to the distal end of the working channel of the ureteroscope. The sheath extends approximately 2 mm from the end of the scope. The fiber tip extends an additional distance beyond the end of the sheath to treat the stones and/or tissue without damaging the sheath or scope.

FIG. 7 demonstrates the exploded view of the components, Universal Luer lock connector and seal (11), that attach to the scope and fiber at the proximal end of the working channel of the scope.

FIG. 8 demonstrates an actual view of the seal and Luer lock connector attached to the stainless steel female Luer lock connector of the ureteroscope.

FIG. 9 shows details of a possible version of the two sets of laser fiber markings that allows re-cleaving of the laser fiber during treatment.

I claim:

1. A protective sheath for an optical fiber used in surgical laser procedures, wherein the protective sheath includes a plurality of holes for improved fluid flow and sterilization.

2. A protective sheath as claimed in claim 1, wherein inner diameter of the sheath is close to a maximum outer diameter of the a buffer of the optical fiber.

3. A protective sheath as claimed in claim 1, wherein an inner diameter of the sheath is larger than an outer diameter of a fiber buffer to allow for introduction of larger diameter devices.

4. A protective sheath for an optical fiber used in surgical laser procedures, further comprising a luer connector for locking the sheath to the working channel of an introducer or scope.

5. A protective sheath for an optical fiber used in surgical laser procedures, further comprising one or more sets of markings at either just the proximal end of the fiber or at both the proximal end of the fiber to facilitate positioning of a distal end of the fiber relative to a distal end of the sheath. The markings at the distal end of the fiber allows the physician to cleave the fiber at the precisely correct position that allows the corresponding mark at the proximal portion of the fiber to be used in positioning the fiber tip recessed into the sheath during insertion and removal from the endoscope.

6. A protective sheath as claimed in claim 5, further comprising trim markings at a distal and/or proximal end of the fiber for facilitating cutting of the sheath to a desired length.

7. A protective sheath as claimed in claim 5, wherein the number of sets of markings on the matched laser fiber is at least two, to facilitate positioning after re-stripping.

8. A protective sheath for an optical fiber used in surgical laser procedures, wherein a proximal end of the sheath is reinforced to facilitate securing of the optical fiber to the sheath by a securing device.

9. A protective sheath as claimed in claim 8, wherein the securing device is a pin vise.

10. A protective sheath as claimed in claim 8, wherein a universal luer lock connector is attached to the reinforced section of the sheath for securing the sheath to an introducer or scope.

11. A protective sheath for an optical fiber used in surgical laser procedures, further comprising a hard stop provided to prevent a laser fiber from extending too far past an end of the sheath.
